(19) United States
(12) Patent Application Publication
(10) Pub. No.: US 2024/0404065 A1
(43) Pub. Date: Dec. 5, 2024

(54) Segmentation Of Bony Structures

(71) Applicant: Stryker Corporation, Kalamazoo, MI (US)

(72) Inventors: Thomas Joseph Gibbons, Manchester (GB); Zygmunt Bispham Jones, Manchester (GB); Graham Richard Vincent, Greater Manchester (GB); Raquel Rodriguez Martin, Manchester (GB)

(73) Assignee: Stryker Corporation, Kalamazoo, MI (US)

(21) Appl. No.: 18/680,414

(22) Filed: May 31, 2024

Related U.S. Application Data
(60) Provisional application No. 63/505,466, filed on Jun. 1, 2023.

(51) Int. Cl. G06T 7/11; G06T 7/00; G06T 7/70; G06V 20/70; G16H 30/40

(52) U.S. Cl. CPC G06T 7/11; G06T 7/0012; G06T 7/70; G06V 20/70; G16H 30/40; G06T 2207/20084; G06T 2207/30012

(57) ABSTRACT

A method for performing segmentation on CT image data of a spine is provided. The method includes retrieving the CT image data of the spine, detecting an estimated position of at least four pedicle regions associated with a plurality of vertebras, determining a pose for each of at least two vertebrae. The method further includes performing a first segmentation process on the CT image data with a shape model to generate a first segmentation and performing a second segmentation process on the CT image data using a first neural network to generate a second segmentation of the at least two vertebrae. The method also includes mapping the shape model to the second segmentation, applying landmarks from the shape model to the second segmentation using the mapping, and overlaying a segmentation mask based on the second segmentation over the CT image data.

### CROSS-REFERENCE TO RELATED APPLICATIONS

The present application is a which claims priority to and all the benefits of U.S. Provisional Patent Application No. 63/505,466, filed on Jun. 1, 2023, the entire contents of which are expressly incorporated herein by reference.

### TECHNICAL FIELD

The present disclosure relates generally to systems and methods related to the segmentation of three-dimensional images of a patient.

### BACKGROUND

Segmentation is an important processing step in many imaging applications for analyzing or interpreting an image. In particular, image segmentation is the process of assigning a label to every pixel in an image such that pixels that share similar characteristics such as color, intensity or texture are given the same label. In medical imaging, segmentation methods are used to identify boundaries of specific objects such as bones, heart, tumors or any such anatomical structures or abnormal regions in a medical image. The medical images may be obtained by means of Magnetic Resonance Imaging (MRI), Computed Tomography (CT), Ultrasound (US) or any other imaging modalities. Identification of specific objects in a medical image helps in extracting meaningful quantitative information from the medical image that will aid in the diagnosis of a patient's medical condition. Segmentation of medical images is also useful for many other applications, including surgical planning, robotic surgery, patient-specific instrumentation (PSI), and drug trials.

Various computer-aided segmentation methods have been developed for segmenting medical images. These methods can generally be classified as automatic (unsupervised), interactive (semi-supervised), and manual (supervised) methods. Manual segmentation methods give reliable results when identifying objects from an image for a particular clinical task, such as tumor detection. In manual segmentation methods, a user or an operator, who is generally a medical practitioner with a knowledge of the human anatomy, utilizes mouse-based software to outline or label regions of specific objects in an image that can be further utilized for providing a specific diagnosis. Such a method of manually identifying specific objects does not serve the needs of daily clinical use well as it is tedious, time consuming and suffers from problems related to operator variability.

Unsupervised or automatic segmentation methods such as ones based on thresholding, watershed, edge detection, morphological operation, neural network, region growing, graph cuts or shape analysis provide segmentation results without prior knowledge about the images and without any user interaction. Unsupervised segmentation methods are generally applied for segmentation of well-circumscribed objects in an image. When applied to medical images, they are able to generate rough segmentation results which would require further refinement.

The shortcomings of automatic and manual segmentation methods led to the development of interactive or semi-automatic segmentation methods. Interactive segmentation methods use a combination of human experts and machine intelligence to improve segmentation accuracy and efficiency. The present application discloses a semi-automatic segmentation method that utilizes landmarks to provide more accurate results compared to prior methods.

### SUMMARY

This Summary introduces a selection of concepts in a simplified form that are further described below in the Detailed Description below. This Summary is not intended to limit the scope of the claimed subject matter nor identify key features or essential features of the claimed subject matter.

According to a first aspect, a method for performing segmentation on image data including a first bone is provided. The method includes retrieving the image data of the first bone, performing a first segmentation process on the image data associated with the first bone with a first shape model to generate a first segmentation of the first bone, and performing a second segmentation process on an image region of the image data associated with the first bone using a first neural network to generate a second segmentation of the first bone. The second segmentation process utilizes, as a first input, the image data associated with the at first bone, and as a second input, the first segmentation of the first bone. The method further includes mapping the output of the first shape model to an output of the second segmentation process and determining the anatomical landmarks in the output of the second segmentation based on the mapping.

According to a second aspect, a non-transitory computer readable storage medium having stored therein data representing instructions executable by a programmed processor for vertebra segmentation for three-dimensional computed tomography is provided. The storage medium includes instructions for: retrieving the CT image data of the spine, performing a first segmentation process on the image data associated with the first bone with a first shape model to generate a first segmentation of the first bone, and performing a second segmentation process on an image region of the image data associated with the first bone using a first neural network to generate a second segmentation of the first bone. The second segmentation process utilizes, as a first input, the image data associated with the first bone, and as a second input, the first segmentation of the first bone. Finally, the storage medium also includes instructions for mapping the first shape model to an output of the second segmentation process and displaying the output of the second segmentation process.\

According to a third aspect, a method for performing segmentation on CT image data of a spine is provided. The method includes retrieving the CT image data of the spine, the CT image data including image data associated with a plurality of vertebrae. After receiving the CT image data, the method further includes detecting an estimated position of at least four pedicle regions for the CT image data associated with the plurality of vertebrae, determining a pose for each of at least two of vertebra based on the detected estimated position of the at least four pedicle regions using on a multiple hypothesis approach, performing a first segmentation process on the CT image data associated with the at least two vertebra with a shape model to generate a first segmentation of the at least two vertebra, and performing a second segmentation process on the image region of the CT image data associated with the at least two vertebra using a first neural network to generate a second segmentation of the at least two vertebra. The second segmentation process utilizes, as a first input, the CT image data associated with the at least two vertebrae, and as a second input, the first segmentation of the at least two vertebrae. The method also includes mapping the shape model to the output of the second segmentation process, applying landmarks from the shape model to the output of the second segmentation process using the mapping, and overlaying a segmentation mask based on the second segmentation over the CT image data.

According to a fourth aspect, a non-transitory computer readable storage medium having stored therein data representing instructions executable by a programmed processor for vertebra segmentation for three-dimensional computed tomography is provided. The storage medium includes instructions for: retrieving the CT image data of the spine, the CT image data including image data associated with a plurality of vertebrae; detecting an estimated position of at least four pedicle regions for the CT image data associated with the plurality of vertebrae; and determining a pose for each of at least two of vertebra based on the detected estimated position of the at least four pedicle regions using on a multiple hypothesis approach. The storage medium further includes instructions for: performing a first segmentation process on the CT image data associated with the at least two vertebra with a shape model to generate a first segmentation of the at least two vertebra; performing a second segmentation process on the image region of the CT image data associated with the at least two vertebra using a first neural network to generate a second segmentation of the at least two vertebra, the second segmentation process utilizing, as a first input, the CT image data associated with the at least two vertebra, and as a second input, the first segmentation of the at least two vertebra. Finally, the storage medium includes instructions for overlaying a segmentation mask over the CT image data, the segmentation mask being based on the second segmentation.

According to a fifth aspect, a method for performing segmentation on image data of a spine is provided. The method starts by retrieving the CT image data of the spine, the CT image data including a first image data associated with a plurality of vertebrae and a second image data associated with at least two of the plurality of vertebrae. The method further includes determining that metal is present in the second image data and employing a metal process in response to determining that metal is present in the second image data. The metal process includes converting at least a portion of the second image data at least one binary image, generating a metal segmentation of the at least two of the plurality of vertebrae using a neural network, and fitting a binary shape model to the at least one binary image. Finally, the method includes overlaying a first segmentation mask over the CT image data, the first segmentation mask being based on the metal segmentation.

According to a sixth aspect, a method for performing segmentation on CT image data is provided. The method begins with retrieving the CT image data, the CT image data including a first image data associated with an anatomical element of a patient and a second image data associated with at least a portion of the anatomical element. Subsequently, the method includes determining that metal is present in the second image data and employing a metal process in response to determining that metal is present in the second image data. The metal process includes converting at least a portion of the second image data from a CT image format to at least one binary image, generating a metal segmentation of the at least two of the plurality of vertebrae, and fitting a binary shape model to the at least one binary image. Finally, the method includes overlaying a first segmentation mask over the CT image data, the first segmentation mask being based on the metal segmentation.

Any of the above aspects can be combined in part or in whole with any other aspect. Any of the above aspects, whether combined in part or in whole, can be further combined with any of the following implementations, in full or in part.

In some implementations, the method further includes detecting an estimated position of at least one region or feature of the first bone in the image data and determining a pose for each of the first bone based on the detected estimated position of the at least one region or feature.

In some implementations, the method further includes applying anatomical landmarks from the first shape model to the output of the second segmentation process using the mapping. In some implementations, the method further includes applying landmarks from the binary shape model to the first segmentation mask using the fitting of the binary of the shape model.

In some implementations, the first bone is a femur, a tibia, a pelvis, or a vertebra. In some implementations, the first bone is a first vertebra and the second bone is a second vertebra which is different from the first vertebra. In some implementations, the first bone is a femur and the second bone is a tibia.

In some implementations, the method further includes retrieving image data of a second bone, performing a third segmentation process on the image data associated with the second bone with a second shape model to generate a first segmentation of the second bone, and performing a fourth segmentation process on the image region of the image data associated with the second bone using a second neural network to generate a second segmentation of the second bone. The fourth segmentation process utilizes, as a first input, the image data associated with the second bone, and as a second input, the first segmentation of the second bone. In some implementations, the method further includes mapping the second shape model to an output of the fourth segmentation process for the second bone and displaying the output of the fourth segmentation process.

In some implementations, the shape model includes at least two model vertebrae, and the step of performing the first segmentation process on the CT image data associated with the at least two vertebrae with the shape model to generate the first segmentation of the at least two vertebra includes associating each of the at least two vertebrae with a respective one of the at least two model vertebrae. In some implementations, the shape model further includes model pedicle positions associated with each of the at least two model vertebrae and each of the at least two vertebrae are associated with the respective one of the at least two model vertebrae based on a comparison of the estimated positions of the at least four pedicle regions and the model pedicle positions. In some implementations, the shape model is a binary active appearance model. In some implementations, the method further includes selecting the shape model from a plurality of active appearance models based on a user input. In some implementations, the first shape model is different from the second shape model.

In some implementations, the first shape model is further defined as a grid of a plurality of active appearance model instances. In some implementations, the method further includes running each active appearance model instance against the image data, culling at least one instance from the plurality of run active appearance model instances based on a cost associated with each active appearance model instance, and performing the first segmentation process using the at least one of the active appearance model instances that remain after the step of culling at least one instance.

In some implementations, the step of determining the pose of the at least two vertebra includes determining the pose of a first vertebra of the least two vertebra relative to a reference coordinate space of the CT image data. In some implementations, the step of determining a pose of the at least two vertebra includes determining a pose of a second vertebra of the at least two vertebra relative to a position and orientation of the first vertebra of the at least two vertebra. In some implementations, the multiple hypothesis approach is a Bayesian multiple hypothesis approach. In some implementations, the step of determining the pose for at least two vertebra based on the detected estimated position using the Bayesian multiple hypothesis approach includes generating a probability weighting graph.

In some implementations, the landmarks include at least one of a first pedicle, a second pedicle, a lamina, and a superior endplate. In some implementations, the method further includes calculating anatomical information including a pose of at least one anatomical landmark based on the second segmentation.

In some implementations, the method further includes receiving user input with respect to an image region of the CT image data, the image region corresponding to one of the plurality of vertebrae. In some implementations, the user input is indicative of a desired level label for one of the plurality of vertebrae.

In some implementations, the method further includes generating a plurality of additional labels, each one of the plurality of additional labels being associated with one of the plurality of vertebra of the CT image data. In some implementations, the method further includes generating a plurality of labels, each one of the plurality of labels being associated with one of the first bone and the second bone. In some implementations, the step of generating the plurality of labels is based on the first shape model and the second shape model.

In some implementations, the first neural network is different from the second neural network. In some implementations, the first neural network is a convolutional neural network. In some implementations, the second neural network is a convolutional neural network.

In some implementations, the method further includes converting the segmentation mask to a surface mesh. In some implementations, the method further includes receiving user input with respect to the segmentation mask. In some implementations, the method further includes, in response to the user input with respect to the segmentation mask, indicating at least one of the segmentation is incorrect and a label associated with a vertebra is incorrect.

In some implementations the method further includes performing a first segmentation process on the first image data with a second shape model to generate a first segmentation of the at least two vertebrae and performing a second segmentation process on the first image data using a first neural network to generate a second segmentation of the at least two vertebrae. The second segmentation process utilizes, as a first input, the first image data, and as a second input, the first segmentation of the at least two vertebrae. In some implementations, the method further includes mapping the second shape model to the output of the second segmentation process, applying landmarks from the second shape model to the output of the second segmentation process using the mapping, and overlaying a second segmentation mask over at least a portion of the CT image data, the second segmentation mask being based on the second segmentation. In some implementations, the method includes overlaying a second segmentation mask over the first image data, the second segmentation mask being based on the second segmentation. In some implementations, the step of overlaying the first segmentation mask over the CT image data includes overlaying the first segmentation mask over the second image data.

In some implementations, the method is carried out by instructions stored on the computer readable storage medium.

### BRIEF DESCRIPTION OF THE DRAWINGS

Advantages of the present invention will be readily appreciated as the same becomes better understood by reference to the following detailed description when considered in connection with the accompanying drawings.

FIG. 1 is a perspective view of an exemplary layout of an operating room including at least one surgical instrument assembly and a surgical navigation system for performing a medical procedure on a patient, according to the teachings of the present disclosure.

FIG. 2 is a schematic of an exemplary graphical user interface of a navigation system, the GUI displaying a selected vertebra region selected by the medical professional to be segmented with the navigation system, according to the teachings of the present disclosure.

FIG. 3 is a flowchart illustrating an exemplary segmentation method including a metal process and an error handling process according to the teachings of the present disclosure.

FIG. 4 is a flowchart illustrating steps of the exemplary metal process shown in FIG. 3.

FIG. 5 is a flowchart illustrating steps of the error handling process shown in FIG. 3.

FIG. 6A is a perspective view of an exemplary spine including a selected vertebra region.

FIG. 6B is a perspective view of the selected vertebra region shown in FIG. 6A as well as detected pedicle regions.

FIG. 6C shows the detected pedicle regions shown in FIG. 6B along with estimated vertebra positions detected according to a multiple hypothesis approach.

FIG. 7A is an exemplary vertebra and detected pedicle and lamina positions.

FIG. 7B is a perspective view of the selected vertebra region shown in FIGS. 6A and 6B including a detected spine centerline.

FIG. 7C is an alternative perspective view of the selected vertebra region and the detected spine centerline shown FIG. 7B.

FIG. 8 is a flowchart illustrating an exemplary segmentation method including a multi-start algorithm according to another implementation of the teachings of the present disclosure.

FIG. 9 is a flowchart illustrating steps of the multi-start algorithm shown in FIG. 8.

### DETAILED DESCRIPTION

### I. Example System Overview

Referring to the Figures, wherein like numerals indicate like or corresponding parts throughout the several views, a surgical system 100 including a surgical navigation system 110 and methods for using the same are shown throughout.

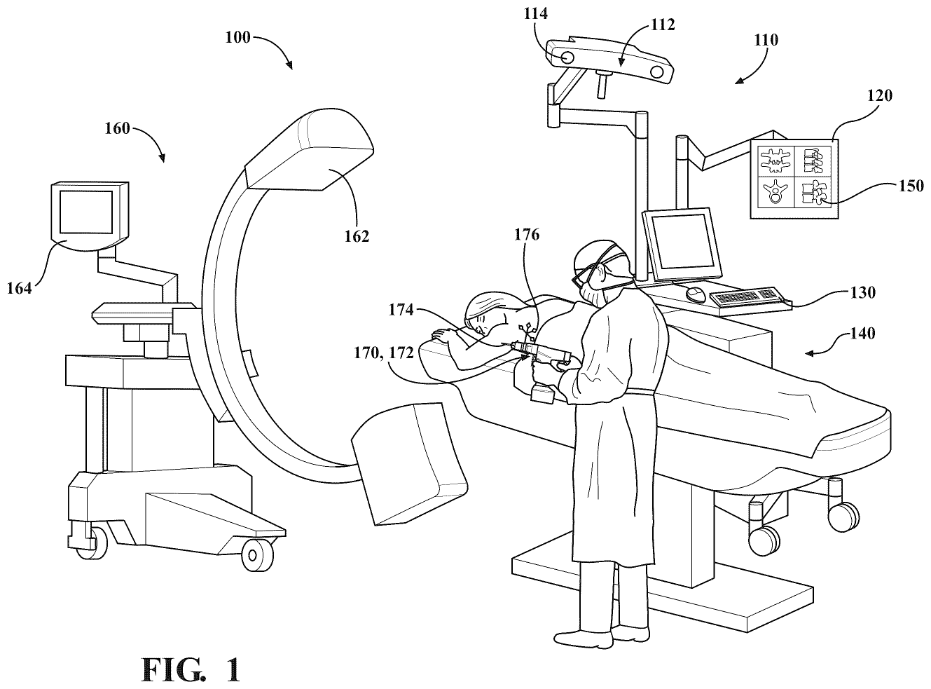

Referring to FIG. 1, an exemplary configuration of an operating room or surgical suite for performing a medical procedure on a patient using the surgical system 100 is shown. The surgical navigation system 110 may include a navigation computer 140, user input devices 130, a display unit 120, and a tracking unit 112. The navigation computer 140 may include a central processing unit (CPU) and/or other processors, memory (not shown), and storage (not shown). The navigation computer 140 may be a personal computer, laptop computer, tablet computer or any other suitable computing device. The navigation computer 140 may include surgical navigation software including one or more modules and/or operating instructions related to the operation of the surgical navigation system 110 and to implement the various routines, functions, or methods disclosed herein.

The display unit 120 is configured to display various graphical user interfaces (GUI) 150 and patient images (e.g., pre-operative patient images or intraoperative patient images). The pre-operative images may be uploaded to the surgical navigation system 110 prior to the surgical procedure. A user such as a medical professional may interact with the various GUIs 150 via user input devices 130 or via touch input. The display unit 120 of the surgical navigation system 110 may be configured to display various prompts or data entry boxes. For example, the display unit 120 may be configured to display a text box or prompt that allows the user to manually enter or select the type of surgical procedure to be performed.

The display unit 120 may be further configured to display a surgical plan for a medical procedure overlaid on the patient images. The surgical plan may include the surgical pathway for executing the medical procedure, planned trajectory, orientation, and/or position for the medical instrument and/or implant during the medical procedure. The surgical plan may also include a pose of an implant or medical device to be inserted during the medical procedure overlaid onto the patient data or image. It is contemplated that the surgical navigation system 110 may be configured to display and/or project a holographic image of surgical pathway for executing the medical procedure or planned trajectory or orientation for the medical instrument during the medical procedure. This may include projecting the surgical pathway onto the patient or other surface in the operating room. It may also include a projection of the surgical pathway onto the head unit worn by the user, such as a lens, shield, or glasses of the head unit. An exemplary configuration of the surgical navigation system 110 including a display unit worn by the user to display the target trajectory and/or target location is disclosed in International Publication No. WO/2018/203304 A1, the entirety of which is hereby incorporated by reference.

The GUI 150 may be configured to allow the user to input or enter patient data or modify the surgical plan. The patient data, in addition to the patient images, may include additional information related to the type of medical procedure being performed, the patient's anatomical features, the patient's specific medical condition, and/or operating settings for the surgical navigation settings. For example, in performing a spinal fusion procedure, the user may enter information via the user input devices 130 and/or the GUI 150 related to the specific vertebra or vertebra on which the medical procedure is being performed. The user may also input various anatomical dimensions related to the vertebrae and/or the size and shape of a medical device or implant to be inserted during the medical procedure. The user input devices 130 and/or the GUI 150 may also be configured to allow the user to select, edit or manipulate the patient data. For example, the user may identify and/or select anatomical features from the patient data. This may include selecting the surgical site, such as selecting the vertebra and/or specific area on the vertebra where the medical procedure is to be performed.

The surgical navigation system 110 may be configured to utilize segmentation to facilitate various features of surgical navigation, such as tool guidance and the generation of alert zones of interests around critical anatomical features. These critical anatomical features may include, cortical walls, nerves, blood vessels or similar critical anatomical structures. The alert zones may be defined by one or more virtual boundaries. The user may also provide input to the user input devices 130 or to the GUI 150 to identify additional critical anatomical features and/or alert zones in addition to what was suggested by the navigation computer 140 or wish to edit alert zones and/or virtual boundaries generated by the navigation computer 140. The user may also provide input to the user input devices 130 or to the GUI 150 to select and/or input a target location, target trajectory, target depth or similar feature of the surgical pathway to help guide the user in performing the medical procedure.

The input to the user input devices 130 or to the GUI 150 may be provided to select the surgical instrument to be used, to select the device and/or implant to be inserted, to select a planned pose where the device or implant is to be placed within the patient, and to allow the user to select the parameters of the implant to be inserted, such as the length and/or diameter of the screw to be inserted. As will be described in more detail in Section II, the input to the user input devices 130 or to the GUI 150 may also affect the segmentation process.

The surgical system 100 may also include an imaging system 160 in communication with the surgical navigation system 110. The imaging system 160, such as CT or MRI imaging device, may perform intraoperative imaging. If the imaging system 160 is a CT imaging device, the imaging system 160 may generate CT image data. The imaging system 160 may include a scanner 162 and a display unit 164. The scanner 162 may be utilized to take an image of the patient and display it on the display unit 164. For example, the scanner 162 may include a C-arm configured to be rotated about the patient to produce a plurality of images of the patient. The imaging system 160 may also include a processor (not shown) including software, as is known by those skilled in the art, which is capable of taking the plurality of images captured by the scanner 162 and producing a 2D image and/or a 3D model of at least a portion of the patient. The display unit 164 may be configured to display the resulting 2D image and/or 3D model.

The imaging system 160 may also be in communication with the navigation computer 140 of the surgical navigation system 110. The imaging system 160 may be configured to communicate via a wired and/or a wireless connection with the navigation computer 140. For example, the imaging system 160 may be configured to provide pre-operative and/or intra-operative image data, such as the resulting 2D image and/or 3D model of the patient, to the navigation computer 140 to provide the resulting 2D image and/or 3D model to the display unit 120. If the imaging system 160 is a CT imaging device, the imaging system 160 may provide the navigation computer 140 with CT image data.

The surgical system 100 also includes a surgical instrument assembly 170 in wired or wireless communication with the navigation computer 140 directly, or indirectly. While only the first surgical instrument assembly 170 is illustrated in FIG. 1, it should be understood that it is only an exemplary configuration of the surgical system 100, and that it is contemplated that any number of surgical instrument assemblies may be positioned within the operating room. The surgical instrument assembly 170 includes a surgical instrument 172 including an end-effector 174 and a tracking device 176. The tracking device 176 includes a plurality of markers that are capable of being identified and/or tracked by the surgical navigation system 110. Reliable tracking of surgical instruments during the execution of surgical procedures to follow the planned surgical pathway and/or to avoid critical anatomical structures is of the utmost importance. Furthermore, providing feedback and/or notifying the user executing the procedure when the surgical instrument becomes misaligned with the surgical pathway and/or is at risk of impinging on a critical anatomical structure is of similar importance. The surgical instrument 172 may be coupled to a drill chuck, a tap for creating threads on the interior surface of a hole or aperture, a driver for driving or inserting a screw within the borehole or aperture of the bone, or another end effector. The surgical instrument assembly 170 may each be like any of those described in Intl. Patent Publication No. 2021/062373, which is hereby incorporated by reference in its entirety. The surgical system may, in addition or as an alternative to the surgical instrument assembly 170, include a surgical robot, such as the robotic manipulator described in U.S. Pat. No. 11,033,341, which is hereby incorporated by reference.

Further, the navigation system 110 may include the tracking unit 112 to track the instrument assembly 170, the surgical robot, and/or other elements of the surgical system 100. The tracking unit 112 may include one or more sensors 114 for tracking the tracking device 176 of the surgical instrument assembly 170. The sensors may include cameras, such as CCD cameras, CMOS cameras, and/or optical image cameras, magnetic sensors, radio frequency sensors, or any other sensor adapted to detect and/or sense the position of a tracking device 176 of the surgical instrument assemblies 170. Description of a suitable tracking unit, and the various localizers that it can utilize may be found in U.S. Patent Publication No. 2017/0333137, which is hereby incorporated by reference in its entirety.

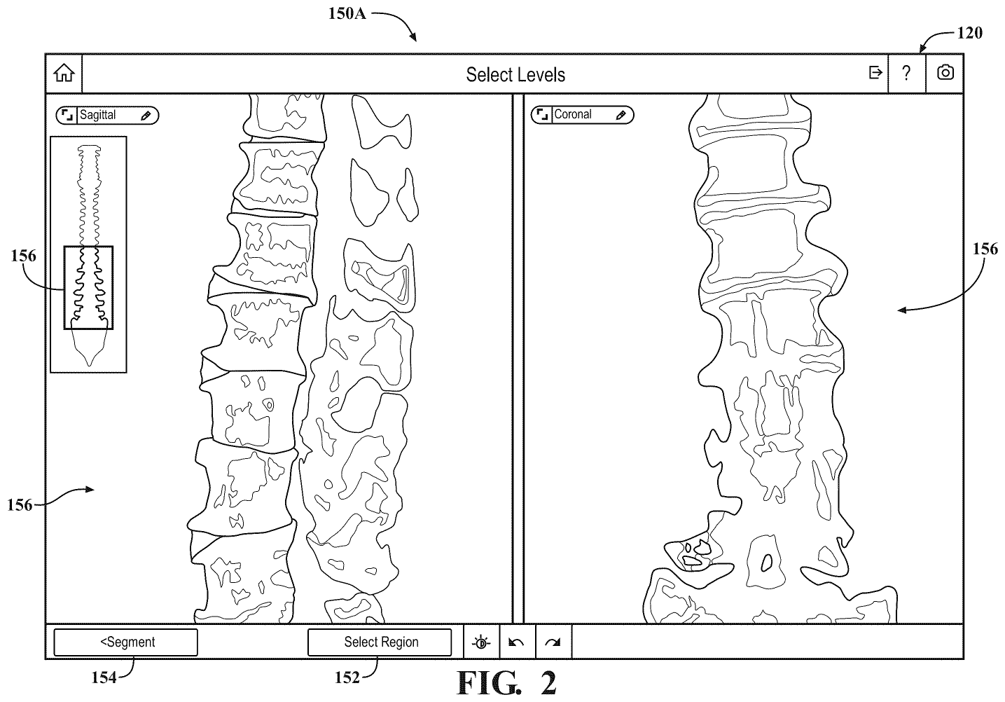

Referring to FIG. 2 an exemplary configuration of a graphic user interface (GUI) 150A of the surgical navigation system 110 is illustrated. The GUI 150A may be configured as a touch screen on the display unit 120 of the surgical navigation system 110. As shown in FIG. 2, the GUI 150A may be referred to as a segmentation interface 150A. The GUI 150A may include a select region button 152 for selecting a region to be segmented and a segment button 154 which the user selects to segment the selected region. The surgical navigation system 110 may be configured to utilize segmentation according to method provided in Section II below to facilitate alert zone planning, tool boundaries, implant position and/or orientation, and other features of surgical navigation. The user may provide input to the user input devices 130 by selecting the select region button 152 of the graphic user interface (GUI) 150A to define a region of interest (e.g. a selected vertebra region 156) of the patient images when the operation is targeted at the selected vertebrae and thus the segmentation of the patient images may be limited to the selected vertebra region 156. Once the user has selected the region of interest, such as the selected vertebra region 156, the user may select a segment button 154 to segment the selected vertebra region 156. Exemplary GUIs and segmentation tools are described in U.S. Patent Publication No. 2019/0340765, which is hereby incorporated by reference.

### II. Exemplary Segmentation Method

As described in Section I above, the surgical navigation system 110 may utilize segmented image data to carry out various functions of the system 110. For example, the segmented image data may be used to identify boundaries of specific objects such as bones, heart, tumors or any such anatomical structures or abnormal regions in the image. The identified objects may be used to extract quantitative information from the image that may be used in the diagnosis of a patient's medical condition, surgical planning, robotic surgery, patient-specific instrumentation (PSI), and drug trials. It will be appreciated that the accuracy of the boundaries of the one or more object portions identified in the segmentation affects the usefulness of the image segmentation. For example, an error in the segmentation can lead to segmented images which do not accurately reflect the anatomy of the patient. The segmented image data may be generated according to segmentation method described herein.

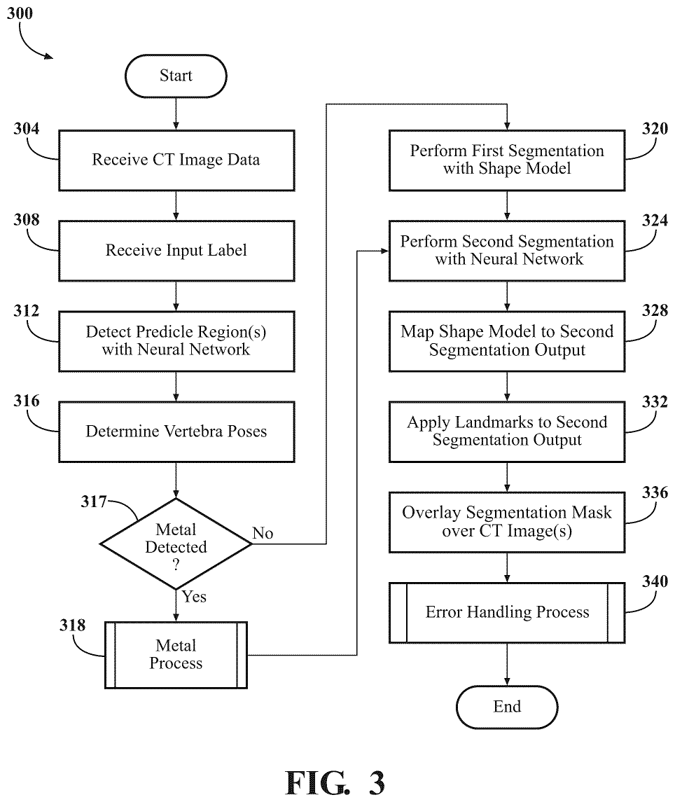

Referring to FIGS. 3 to 7C, details regarding an exemplary segmentation method are provided. The segmentation method provided herein takes advantage of other technologies, such as neural networks and shape/active appearance models and combines these technologies in such a way that the user is provided with a more accurate segmentation, along with integrated surgical planning. Although the methods are described as being carried out by the navigation computer 140, it will be appreciated that the methods could be carried out by another computing device or a combination of the navigation computer 140 and another computing device. For example, the user inputs and CT image data may be provided to the navigation computer 140, and the navigation computer 140 could communicate the with at least one other computing device in order to carry out the methods described herein. For example, the segmentation method may be at least partially carried out on a dedicated image processing computer. The dedicated image processing computer may be part of the navigation computer 140 or may instead be separate from the navigation computer 140 and in communication with the navigation computer 140. It will also be appreciated the term “shape model” is used herein to include any of an active shape model, an active appearance model, active contour model, or other suitable type of statistical shape model. An exemplary shape model is described in U.S. Pat. No. 7,584,080, which is hereby incorporated by reference in its entirety.

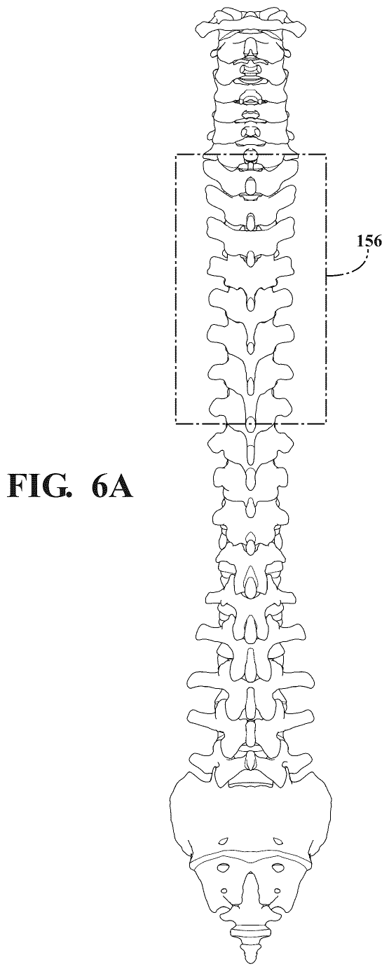

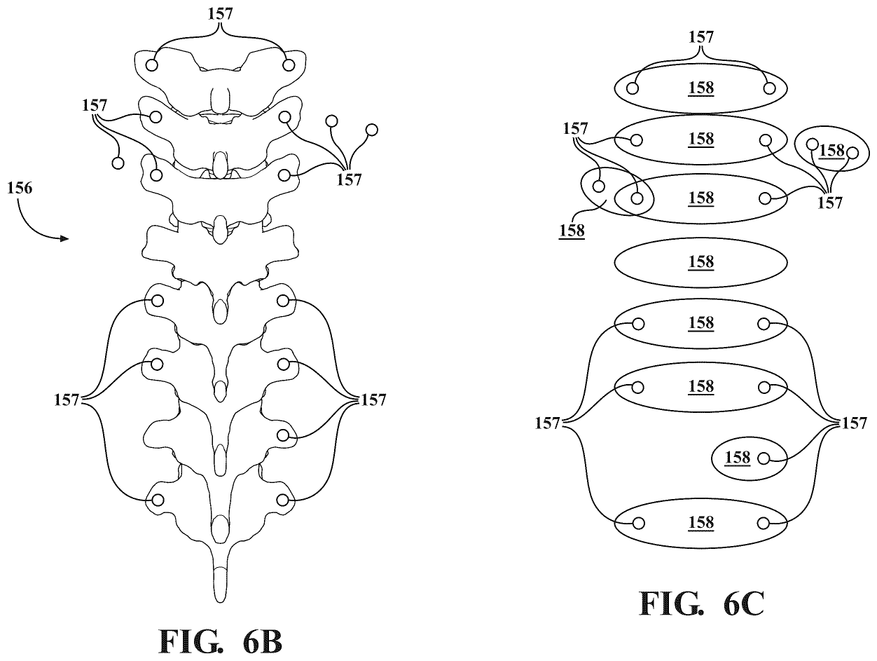

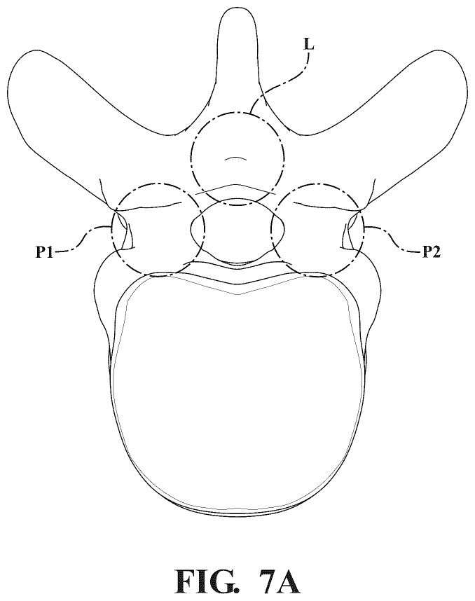

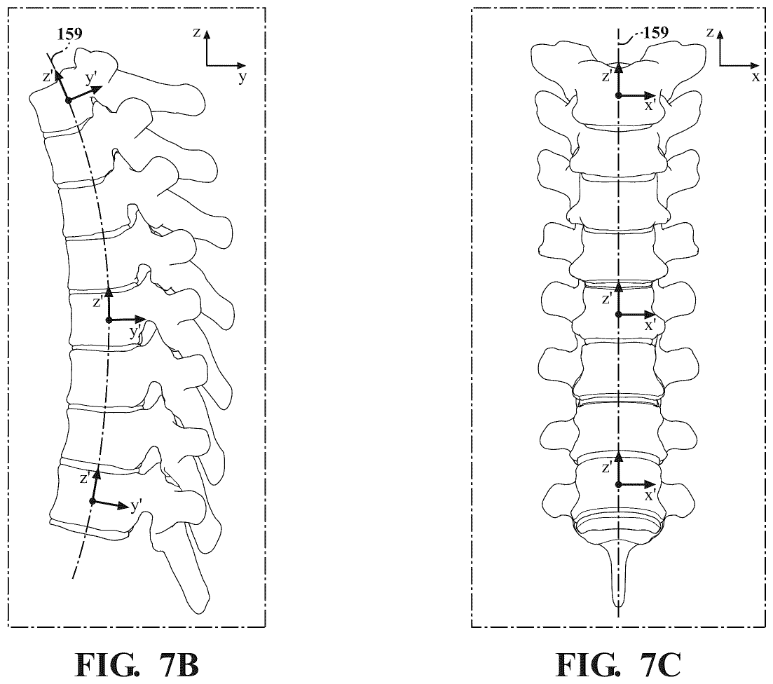

Referring to FIG. 3, a segmentation method 300 according to the teachings of the present disclosure is provided. At 304, image data, such as CT image data, is input to and received by the navigation computer 140. For example, the CT image data may include at least one CT image corresponding to the patient and may be created by imaging system 160. Further, the CT image data includes an anatomical structure, such as a spine, or a portion of the spine, of the patient. The spine or portion of spine included in the CT image data includes at least two vertebrae. At 308, the user selects a vertebral level using one or more of the user input devices 130, the selected vertebral level indication the starting vertebra of the method 300. The selected level is then input to and received by the navigation computer 140. At 312, the navigation computer 140 detects points/voxels of the image data which likely correspond to pedicles of the patient's spine. In other words, the navigation computer 140 attempts to detect an estimated position for each of a plurality of pedicles of the patient's spine present in the image data. The navigation computer 140 may utilize a neural network to detect the pedicle regions present in the image data. The neural network may be a convolutional neural network or any suitable alternative. It is also contemplated that the user may select the vertebral level after 308 but prior to a first segmentation as described below. It is also contemplated that the computer may use one or more techniques other than a neural network to detect the pedicles, such as random forests, support vector machines, and/or k-means algorithm. At 316, the navigation computer 140 attempts to determine a pose of each of the vertebrae present in the image data based on the estimated positions of the plurality of pedicles. The pose of a vertebra generally includes the position and orientation of the vertebra. The navigation computer 140 may utilize a multiple hypothesis approach to determine the pose of each of the vertebrae present in the image data. The multiple hypothesis approach may be a Bayesian multiple hypothesis approach or any other suitable alternative. When the navigation computer 140 determines estimated positions of the plurality of pedicles, the computer 140 receives a plurality of points each corresponding to the estimated position of one of the pedicles and attempts to group and correlate these points to corresponding vertebrae. Further details regarding determining the pose of the vertebrae as well as the multiple hypothesis approach are described in reference to FIGS. 6A-7C below.

At 317, the navigation computer 140 optionally determines if any metal is present within the image data. When the image data is created by the imaging system 160 (e.g. CT scanner 162), x-rays are passed from an x-ray emitter (located on one side of the patient), through the patient, and received by an x-ray detector (located on an opposing side of the patient) to generate a CT image. The scanner 162 then rotates, another CT image is generated, and the process repeats as the scanner 162 rotates about the patient. Normally these x-rays are attenuated to various degrees as the x-rays pass through tissues of varying densities, and the resulting image data contains a 3D image consisting of a large number of voxels (3D pixels). Each voxel is colored according to the intensity of the x-ray(s) received by the x-ray detector after passing through the patient. This intensity is measured in Hounsfield units (HU). However, metal within the patient (e.g. a spinal implant) may be dense enough to attenuate the x-rays to an extreme degree. As a result, the image data will contain voxels with extremely high intensity values (or extremely low intensity values depending on how the imaging system 160 is configured) wherever metal was present during the scan.

In light of the above, the navigation computer 140 can determine where metal, if any, is present in the image data by looking to the intensity values of the voxels in the image data. In one implementation, the navigation computer 140 sets a voxel intensity threshold and determines which voxels have an intensity value over the threshold. The navigation computer 140 then identifies these voxels as likely containing metal. To reduce false-positives, or to otherwise improve the results of the metal detection, the navigation computer 140 may further set a voxel group intensity threshold and determine which groups of voxels contain a threshold number/concentration of voxels which have an intensity value over the voxel intensity threshold. The navigation computer 140 then identifies these groups of voxels as likely containing metal.

If the navigation computer 140 identifies metal in the image data based on the above, the segmentation method 300 proceeds to a metal process at 318. Otherwise, the segmentation method 300 proceeds to 320. The segmentation method 300 employs a first segmentation process at 320 as described below, which includes fitting at least one shape model to the image data. This fitting process may not be reliable if metal is present in the image data, and the segmentation method 300 includes a separate process for CT images containing metal. This process is herein referred to as the metal process.

In some implementations, and as also described below, the first segmentation is carried out on each detected vertebra individually. In other words, shape models each containing a single vertebra are matched to the corresponding detected vertebra. In such an implementation, the metal process may be carried out on only the vertebra(e) which contain metal while the other detected vertebra(e) (which do not contain metal) are segmentation with the first segmentation process. Alternatively, if the first segmentation is carried out on two or more of the detected vertebrae, the metal process may be utilized if at least one of the detected vertebrae contain metal. At 318, the metal process is called by the navigation computer.

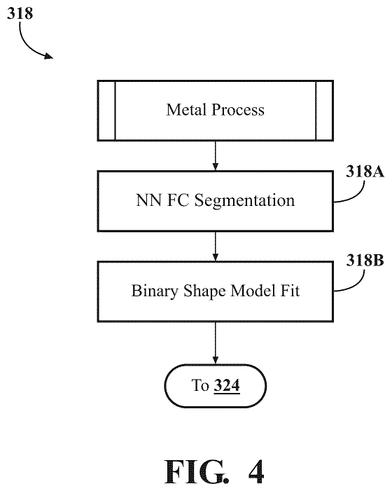

Referring to FIG. 4, the metal process is illustrated along with subprocesses included in the metal process. As noted above, the process of fitting at least one shape model to the image data may be unreliable where metal is present. In order to increase reliability of the segmentation method, the navigation computer 140 can covert at least a portion of the image data (e.g. a detected vertebra containing metal) from CT image format to at least one binary image and fit the at least one shape model to the at least one binary image. It will be appreciated that the metal process may be carried out on only the detected vertebra which contain metal, any subset of detected vertebra in which metal is present, or all of the detected vertebrae if at least one vertebra contains metal. For clarity and ease of description, the forthcoming description of steps 318A and 318B assumes that only one detected vertebra contains metal and that the metal process is carried out on this one detected vertebra. It is contemplated that more than one, or even all, detected vertebra may contain metal and that the metal process may need to be carried out on any number of detected vertebrae.

At 318A, the navigation computer 140 converts at least a portion of the image data (e.g. a detected vertebra containing metal) from CT image format to at least one binary image. The navigation computer 140 generally utilizes a fully connected neural network to carry out this conversion. In an implementation where the navigation computer 140 detects one vertebra with metal and where step 318A is carried out on only that one vertebra, the navigation computer 140 (i.e. the fully connected neural network) starts by splitting the image data into “cubes” and defining local coordinate systems for each of these cubes. Each cube generally corresponds to a section of the image data. More specifically, as the image data is a volumetric image with a height and a width, each cube may be a section of the image data. In some implementations, each cube corresponds to a single vertebral level. In other implementations, each cube corresponds to multiple vertebral levels. In either case, the navigation computer 140 also defines a local coordinate system for each cube when splitting the image data. After splitting the image data into the cubes, the navigation computer 140/neural network can segment each cube separately. This allows the navigation computer 140 to carry out the metal process on any cubes which contain metal. The navigation computer 140 further converts each cube containing metal into binary image(s).

At 318B, the navigation computer 140 fits a binary shape model to the binary image(s) to create a metal segmentation. After the binary shape model is fit, the segmentation method 300 proceeds to 324.

Referring back to FIG. 3, at 320, the navigation computer 140 performs the first segmentation process on the image data. The first segmentation process includes generating a first segmentation of the vertebrae using the output of the multiple hypothesis approach as well as at least one shape model. In one implementation, the first segmentation is generated by matching each of the detected vertebra with an existing shape model of a single vertebra. This matching is performed by determining which shape model best matches each vertebra according to the pose of each vertebra determined at 316. In such an implementation, the existing shape models may only contain the shape of one vertebra as opposed to the shape of the entire spine. This means that the shape model that matches one of the detected vertebrae may be specific to one detected vertebra. In another implementation, the shape model contains the shape of at least two vertebra and may even contain the shape of the entire spine. Here, the matching is performed by determining which shape model best matches the shape of the patient's spine according to: the pose of each vertebra determined at 316 compared to the pose of each corresponding vertebra (e.g. a pose of an T2 vertebra of the patient compared to a pose of an T2 vertebra included in the shape model), an aggregate pose of two or more vertebra determined at 316 compared to an aggregate pose of two or more corresponding vertebra included in the shape model, and/or a combination thereof. In yet another implementation, the user provides an input to the navigation computer 140 which influences the matching. For example, the user may select a shape model from a plurality of shape models to match to the detected vertebra(e).

In any implementation, the shape model may be realized as a plurality of shape model instances such that the plurality of shape model instances is utilized in place of the singular shape model. Multiple pluralities of shape model instances may also be used. For example, where the first segmentation is generated by matching each of the detected vertebra with an existing shape model of a single vertebra, the first segmentation may be generated by matching each of the detected vertebra with one plurality of shape model instances, respectively. In such implementations, the first segmentation process includes generating a first segmentation of the vertebrae using the output of the multiple hypothesis approach as well as the plurality of shape model instances. Further, the method 300 may include determining a cost associated with each shape model instance and culling at least one instance from the plurality of shape model instances based on the cost(s). Subsequently, the first segmentation process is performed using at least one of the shape model instances of the plurality of shape model instances which remains after culling at least one instance.

The navigation computer 140 is capable of matching a shape model to a detected vertebra based on at least one of (1) the selected vertebral level or (2) the details of the shape model known to the navigation computer 140. If relying on selected vertebral level (provided by the user at 308), the navigation computer 140 determines the specific level (e.g. T3) of each vertebral level by propagating the vertebral level through the rest of the vertebral levels present in the image data. For example, if the user provided a label of T2 on the uppermost vertebra in the image data, the computer 140 may label the second uppermost vertebra T3, the third T4, and so on. After doing so, the computer 140 may then match a shape model to each detected vertebra based on the level label. For example, the computer 140 may match a shape model containing a T3 vertebra to the vertebra labeled as T3 in the image data. Alternatively, the computer 140 may match a shape model according to the details of the shape model know to the computer 140. For example, the navigation computer 140 may know the precise location of each pedicle of the vertebra as well as a lamina of the vertebra present in the shape model based on information associated with the shape model. Thus, the navigation computer 140 can match each of the detected vertebra to an appropriate shape model by matching the relative orientation/location of the pedicles/lamina of each detected vertebra present in the image data with the shape model that has substantially similarly oriented/located pedicles/lamina. In another example, the navigation computer 140 may compare the shape of the outer surface of the shape model to the shape of the outer surface of the vertebra in the image data. These methods apply where the shape model(s) contains a single vertebra as well as where the shape model(s) contains two or more vertebrae.

At 324, the navigation computer 140 performs a second segmentation process on the image data. The second segmentation process is configured to refine the first segmentation and includes generating a second segmentation of the vertebrae by providing two inputs to a neural network: the image data, and the first segmentation of the vertebrae. Alternatively, the second segmentation process may refine the metal segmentation by generating the second segmentation of the vertebrae by providing the image data and the metal segmentation to the neural network. In either case, the neural network used at 324 may be the same or different from the neural network used at 312. Either neural network may be a convolutional neural network or any suitable alternative. At 328, the navigation computer 140 fits the shape model to the second segmentation. In one implementation, the computer 140 morphs the shape of the shape model until it is substantially similar to the second segmentation. For example, if the method 300 is being carried out on a T3 vertebra, the shape model includes a model T3 vertebra. This model T3 vertebra is spatially morphed to be the same shape as the T3 vertebra in the image data. At 332, the navigation computer 140 applies landmarks (e.g. pedicle locations, lamina locations, superior endplate locations, etc.) of the shape model to the second segmentation. The locations of the landmarks of the shape model are aligned with the locations of the landmarks in the second segmentation when the model is morphed to fit the second segmentation. At 332, these landmark locations are applied to the second segmentation itself. For example, if the landmark is a pedicle of the model T3 vertebra described above, this T3 pedicle landmark is applied to the second segmentation at substantially the same location as the T3 pedicle. The shape model may include other landmarks which are also applied to the second segmentation.

As described above, the navigation computer 140 provides the image data and the first segmentation of the vertebrae to the neural network in order to generate the second segmentation. The image data is generally input to the neural network in a format that is substantially similar to the standard CT image format (voxel intensities and/or Hounsfield Units). The first segmentation, on the other hand, may be input to the neural network as either a signed distance function or an isosurface (also called a partvol). Regardless of the implementation, the first segmentation is converted from a mesh format to an image format prior to being input to the neural network. This way, the two inputs, the CT image and the first segmentation, are provided to the neural network in a common format (a volumetric image format). Other formats are contemplated.

The shape model may also integrate planning information, such as the position, orientation, or size of a planned implant or surgical instrument. Examples of integrated planning information are described in U.S. Publication No. 20090089034, which is hereby incorporated by reference. Alternatively, the shape model may integrate zones that define alert zones to provide feedback to users. Exemplary zones are described in US20220338938, which is hereby incorporated by reference in its entirety.

Also, the planning information is incorporated into the shape model by defining the planning information in terms of a number of points. It will be apparent that other combinations of points can be used in order to define the planning information and that different types of planning information can be incorporated. In the following examples, the planning information relates to the position, orientation and size of surgical implants. However, the planning information does not need to be limited to, or include all or, position, orientation or size. Further, the planning information can relate to different types of components and is not limited to implants. For example, the planning information can relate to the position, orientation, size or type of instruments, tools or other implements used during a surgical procedure.

Orientations are represented by two points, with the straight line there through defining the orientation. Orientations can also be derived from combinations of points by carrying out geometric calculations using the points obtained from an instantiation of the model. For example, a plane may be defined by three points and then an orientation be derived from the plane as being the direction normal to the plane. Similarly angles may be derived from the angle subtended by the intersection of two straight lines passing through points obtained from the shape model or from the angle subtended by the intersection of a straight line passing through two points from the model and a plane passing through three points obtained from the model.

At 336, the navigation computer 140 overlays a segmentation mask, the mask being based on the second segmentation, over the image data. In some implementations, the navigation computer 140 utilizes an algorithm such as marching cubes or the like to convert the segmentation mask into a refined surface mesh. In such an implementation, the navigation computer 140 also projects/maps the shape model (and its corresponding information) onto the refined surface mesh to create a segmentation output. The segmentation output is thus based on the second segmentation and may further include the landmarks, planning information, and/or zones from the shape model. The segmentation mask may be a binary segmentation mask, a semantic segmentation mask, or a suitable alternative. The segmentation mask may be converted into a surface mesh that is aligned with the image data. In one implementation, the segmentation mask is converted into the surface mesh using a neural network and refined with a marching cubes algorithm. As a result, the user may view the surface mesh overlaid onto the image data with the GUI 150.

In some implementations, the segmentation mask is based on both of the second segmentation and the metal segmentation. The navigation computer 140 still overlays the segmentation over the image data and the navigation computer 140 may still utilize an algorithm such as marching cubes or the like to convert the segmentation mask into the refined surface mesh. In such an implementation, however, the navigation computer 140 not only projects/maps the shape model (and its corresponding information) onto the refined surface mesh like in the above implementation, but also projects/maps the binary shape model (and its corresponding information) from the metal process onto the refined surface to create a segmentation output.

At 340, the navigation computer 140 employs an error handling process to determine whether any errors exist within the segmentation mask and/or whether any errors otherwise occurred during the segmentation method 300.

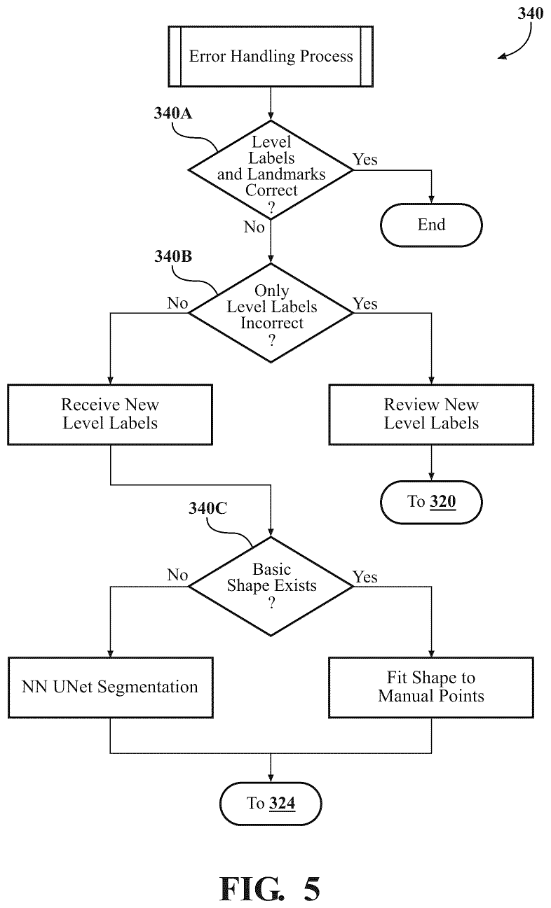

Referring to FIG. 5, the error handling process is shown along with subprocesses included in the error handling process. The error handling process begins at 340A and determines whether the labels of the vertebrae in the selected vertebra region 156 are correct along with whether the landmarks from the shape model were correctly mapped/applied to the image data. In one implementation, the computer 140 automatically determines whether the labels and landmarks were correctly applied to the image data (e.g. via the segmentation mask). As a result of this automatic determination, the computer can determine whether the error handling process should be started. In another implementation, the user may provide an input to the computer 140 to start/control the error handling process. For example, the user may view results of the segmentation method 300 on the GUI 150 and determine that errors in the segmentation mask exist. The user may then provide input to the computer 140 via the GUI 150 to start the process of correcting the segmentation result/mask. In yet another implementation, the computer 140 automatically determines whether any errors are present in the segmentation result and ask the user if the error handling process should be run. The user may then decide whether to start the error handling process or to continue with the existing segmentation result.

If the user and/or the navigation computer 140 determines that both the labels and landmarks of the segmentation output match the image data after the segmentation mask is transformed into the segmentation surface mesh and overlayed over the image data (e.g. at 336), the error handling process ends. If at least one of the labels and landmarks are incorrect, the error handling process continues to 340B. At 340B, the navigation computer 140 determines if only the vertebra labels are incorrect or if both the labels and landmarks are incorrect. If only the labels are incorrect, the navigation computer 140 prompts the user to input corrected level labels for the incorrectly labeled vertebra(e). After receiving the corrected labels, the error handling process directs the navigation computer 140 to 320 to redo steps 320 through 340 of the segmentation method. If, however, the labels and landmarks are both incorrect when compared to the image data, the error handling process prompts the user to input corrected level labels and continues to 340C. At 340C, the navigation computer 140 determines whether the basic shape of the vertebra (now correctly labeled) matches any of the shape models corresponding to the level associated with the vertebra. If no available shape models match the shape of the vertebra, a bone detector network is used. An example of a bone detector network is described in U.S. Pat. No. 7,593,762, which is hereby incorporated by reference in its entirety.

After 340C and the corresponding processes, the segmentation method 300 continues/returns to 324 and performs the second segmentation process on the image data. The method 300 then continues as shown in the figures.

Referring to FIGS. 6A-6C, an image of the patient's spine is depicted along with a plurality of identified points corresponding to possible pedicle locations. As noted above, the first segmentation process is generated, at least in part, using the multiple hypothesis approach. Although the method 300 states that the multiple hypothesis approach is used to determine the pose of the vertebra present in the image data, these poses are simply the end result of the approach.

Referring to FIG. 6A, an exemplary image of the patient's spine is depicted. The spine image is at least a portion of the image data captured by the imaging system 160 and may be viewed by the user via the segmentation interface 150A. In order to facilitate the segmentation method(s) described herein, the user may select the selected vertebra region 156. The image data as received by the navigation computer 140 (e.g. at 304) may thereafter consist only of the image region pertaining to the selected vertebra region 156. For example, FIGS. 6A and 6B show a situation in which the selected vertebra region 156 includes a first thoracic vertebra T1, an eighth thoracic vertebra T8, and those in between. The user may select any region of the patient's spine, and the selected vertebra region 156 may be adjusted to fit the desired range of vertebrae as long as at least two vertebrae are contained within the region 156.

Referring to FIG. 6B, the selected vertebra region 156 is shown. In some implementations, the image data received by the navigation computer 140 may be substantially related to the selected vertebra region 156. In terms of the multiple hypothesis approach and the segmentation method 300, the selected vertebra region 156 may be received by the navigation computer 140 at 304 and further utilized during 308 to 316 (depicted in FIG. 3). As noted above, the user selects a vertebral level at 308 using one or more of the user input devices 130 and the selected vertebral level is communicated to the navigation computer 140 as the starting vertebra for the method 300. In the illustrated implementation, the selected vertebral level is the first the first thoracic vertebra T1. In such an implementation, the segmentation method 300 segments the first thoracic vertebra T1 through the eighth thoracic vertebra T8.

As part of the segmentation method 300, the navigation computer 140 attempts to detect points of the selected vertebra region 156 which correspond to pedicles of the patient's spine. A plurality of estimated pedicle positions 157 are shown in FIGS. 6B and 6C as circles overlaying the selected vertebra region 156. The estimated pedicle positions 157 may be detected/estimated with the neural network employed at 312. It will be appreciated that although the estimated pedicle positions 157 are shown two-dimensionally, these positions 157 are located in 3D image space and are similarly located (i.e. located relative to 3 axes).

Referring to FIG. 6C, the estimated pedicle positions 157 of FIG. 4B are shown without the selected vertebra region 156. FIG. 6C is meant to help provide more detail regarding the multiple hypothesis approach. After the pedicle positions 157 are determined by the navigation computer 140, the computer 140 determines at least one estimated vertebra position 158 based on the estimated pedicle positions 157. More specifically, the navigation computer 140 attempts to determine a plurality of pairs of estimated pedicle positions 157 to determine the at least one estimated vertebra position 158. In the illustrated implementation of FIG. 6C, the navigation computer 140 has determined that up to ten pairs of estimated pedicle positions 157, and thus ten estimated vertebra positions 158, may exist in the selected vertebra region 156. After the estimated vertebra positions 158 have been determined, the navigation computer 140 determines the pose of the vertebra(e) in the selected vertebra region 156.

The multiple hypothesis approach takes the estimated pedicle positions 157 as input and attempts to find the most likely spine shape (i.e. spine centerline) with the inputs 157. The spine shape is found by grouping the estimated pedicle positions 157 to form vertebrae, removing false positive pedicle positions, and estimating locations of missed pedicles positions. The input points 157 are used to produce a one or more reasonable hypotheses that solve the aims listed above. Different combinations of groupings, false positives and misses that fall within reasonable constraints form the hypotheses. The remaining problem is how to find the most likely hypothesis by assigning an approximate probability to each hypothesis.

The probabilities are calculated as a relative probability score with the comparison point being the state where all the detected points 157 are false positive. In this way grouping key points to form a hypothesized vertebra 158 removes the probability of a false positive from the calculation. Hypothesized vertebrae 158 are less likely to be false positives if they have a complete set of points 157 in the correct arrangement while single ungrouped points 157 are less likely but still may be vertebra. Negative log probabilities are used so removing a false-positive decreases the score for a hypothesis. The relative position between two adjacent vertebrae 158 increases the probability score. There is a probability distribution of displacements between adjacent vertebra 158 which can be used to calculate this. Further, there is a probability that one or more vertebra levels have no points 157 detected due to image quality or clinical issues. This is also factored into the calculation. Each hypothesis contains a set of points 157 grouped into vertebrae 158 as well as ungrouped points 157. The multiple hypothesis approach does not arrange the vertebrae into the spine shape. The probability score and the spine shape can be calculated at the same time by formulating each hypothesis into a directed graph and solving using a minimum path solver.

For each hypothesized vertebra 158 and ungrouped point 157 two nodes are defined. One labelled ‘IN’ and one labelled ‘OUT’. An edge is added between every ‘OUT’ node to every ‘IN’ node that is part of a vertebra 158 or point 157. The cost of that edge is calculated as the negative of the log probability of that vertebra-to-vertebra displacement, which gives a positive edge cost. An edge is added from the ‘IN’ to the ‘OUT’ node of every vertebra or ungrouped key point, the cost of this edge is the log probability of the key points being false positives, which results in a negative cost. The minimum path from any to all nodes is calculated using the bellman-ford algorithm initialized from each node in turn. As some nodes' costs are negatives the path length will typically not be zero. The multiple hypothesis approach balances the probability of unlikely vertebra displacements and one or more skipped levels against the probability of correctly arranged key points being false positives.

The adjacent vertebra displacement probabilities require some global parameters. The system starts with loose general parameters for a first pass which only runs on the single most likely hypothesis (as calculated by grouping probabilities only). From this solution the direction and general curvature of the spine is calculated. The second pass runs through all feasible hypotheses with the tuned parameters to find the minimum path across all hypotheses. Once the most likely grouping and path is found the position of missed levels is estimated. If the gap between two adjacent levels large, the expected vertebra spacing calculated from the rest of the spine can be used to estimate if there are one or more missed levels. The number and position of any missed levels can be estimated, and the orientation set to align to a local direction of the spine.

As a result of the multiple hypothesis approach, the navigation computer 140 knows and/or can calculate the location of three points for each detected vertebrae and may determine the pose of each of the detected vertebrae with these points. The three points are described with reference to FIGS. 7A-7C below.

Referring to FIG. 7A, an exemplary vertebra is depicted along with the three points detected by the navigation computer 140 when determining the pose of the detected vertebrae. The three points include a first pedicle P1, a second pedicle P2, and a lamina L. The pedicles P1, P2 are detected at 312 using the neural network as described above with reference to FIGS. 6A-6C. The lamina L may be estimated according to the positions of the pedicles P1, P2 and a spine centerline determined using the multiple hypothesis approach at 316. The processes by which the spine centerline and vertebra poses are determined is further described with reference to FIGS. 7B and 7C.

Referring to FIGS. 7B and 7C, an exemplary spine is depicted along with a detected spine centerline 159 and the detected poses of the vertebrae present in the selected vertebra region 156. As noted above, the poses of the vertebra present in the selected vertebra region 156 may be calculated with the result/output of the multiple hypothesis approach (e.g. the spine centerline 159). In one implementation, each vertebral level initially includes the estimated pedicle positions 157, P1, P2, and the lamina L is calculated using a local direction of the spine. The local direction of the spine can be calculated as a slope of the spine centerline 159 at the center of the vertebral level. As shown in FIGS. 7B and 7C, an image coordinate system ICS is defined in the image data and the poses of the vertebra are defined related to the image coordinate system ICS. More specifically, the poses of the vertebra each include an origin, an x-axis, a y-axis, and a z-axis, each axis extending from the origin. The origin and the x, y, and z axes together form a local coordinate system LCS of the respective vertebra. In order to differentiate the local coordinate system(s) LCS from the image coordinate system ICS, the local coordinate system(s) LCS is depicted with x′, y′, and z′ axes. Poses and local coordinate system(s) LCS are calculated for each detected vertebra. FIGS. 7B and 7C are aligned with the zy-plane and the zx-plane of the global coordinate system GCS, respectively.

The slope of the spine centerline 159 may be calculated for a local vertebral level by first determining a vector extending between the two pedicles P1, P2, to determine a first dimension of the local coordinate system LCS (i.e. the x′-axis). The second dimension of the local coordinate system LCS (e.g. the y′-axis) may be calculated by first determining the slope of the spine centerline 159 at the vertebral level to determine a local spinal direction. The slope may be determined by any mathematical means. In one implementation, the slope is determined by finding a vector spanning from a point on the spine centerline corresponding to a vertebral level above the local vertebral level and to a point on the spine centerline corresponding to a vertebral level below the local vertebral level. For example, for the T2 vertebra, a vector spanning from the T1 vertebra to the T3 vertebra can be used to determine the slope of the spine centerline at the T2 vertebra. Once the local spinal direction is determined, the local spinal direction is treated as the z′-axis of the local coordinate system LCS. This z′-axis extends from the center of the vector extending between the two pedicles P1, P2. The y′-axis of the local coordinate system LCS may then be calculated by taking the cross product of the x′ and y′ axes.

It will be appreciated that although the method is described above with reference to CT image data and the spine of the patient, the method may also be applied to other forms of 3D image data and other tissues of the patient. In one example, the method may utilize MRI image data or the like. In another example, the method may be used to segment a joint of the patient, such as a knee joint, or a hip joint. Other alterations to the method are contemplated. The described system and method may be useful for a variety of orthopaedic joint procedures (for example replacement of hip, knee, shoulder, ankle and elbow joints), peri-acetabular osteotomy, tibial osteotomy, distal radius osteotomy, anterior cruciate ligament reconstruction, osteoid osteoma excision, bone tumor resection, spinal procedures (for example in the placement of pedicle screws), and fracture surgery. To these ends, another implementation of the method is described below.

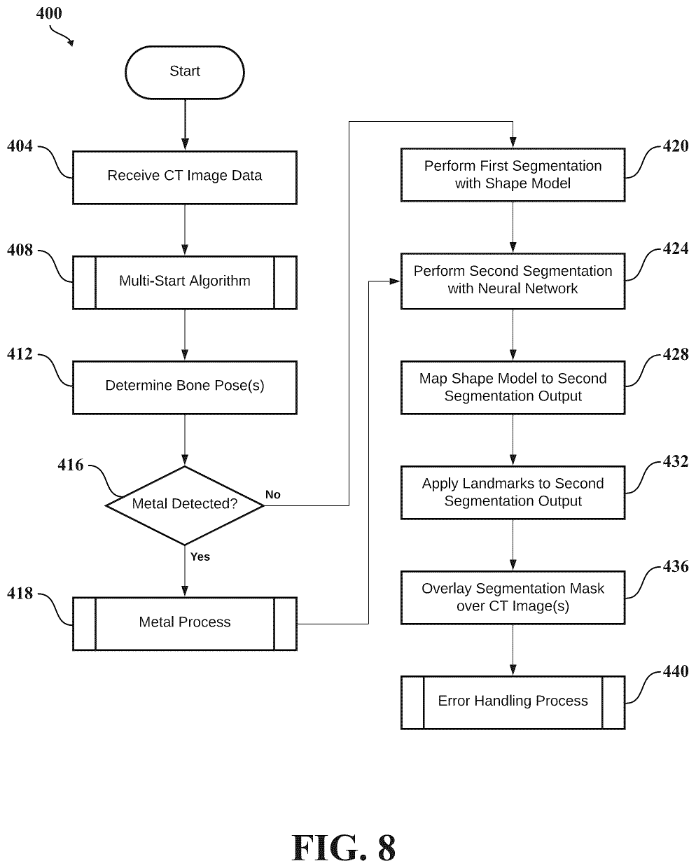

Referring to FIG. 8, a segmentation method 400 according to another implementation is shown. Although the method 400 is described below as being carried out by the navigation computer 140, this is only one implementation of the method 400. Alternatively, the method 400 may be carried out by at least one processor/controller, optionally including the navigation computer 140. Similar to the method 300 described above, at 404, the method 400 begins with the navigation computer 140 receiving image data, such as CT image data. In this implementation, the image data includes an anatomical region of the patient which includes at least one bone, such as a knee or hip region of the patient. After the image data is received, the method 400 proceeds to 408. At 408, a multi-start algorithm is called by the method 400.

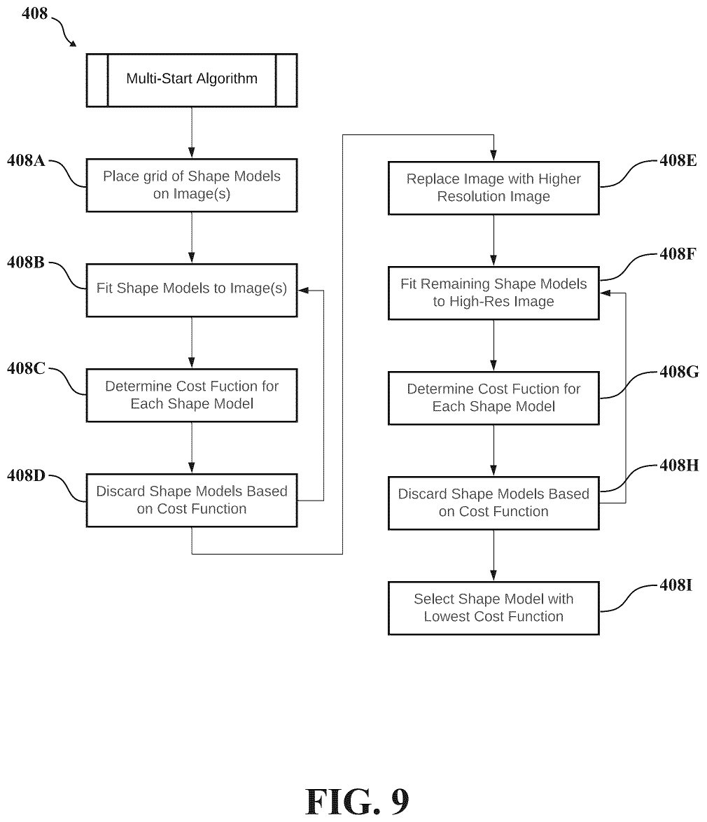

Referring to FIG. 9, a flowchart describing the multi-start algorithm (i.e., the step 408 of the method 400) is illustrated. At 408A, multiple shape models, such as a grid of shape models, is overlaid on the image data. At 408B, the navigation computer 140 attempts to fit each shape model of the grid of shape models to the image data. At 408C, the navigation computer 140 determines a cost function associated with each of the shape models. For example, the cost function associated with a shape model may be a current root-mean-square (RMS) residual between the shape model and the image data. At 408D, the navigation computer 140 discards at least one shape model of the grid of shape models based on the cost function of each shape model. More specifically, the shape model(s) with the highest cost function(s) may be discarded. The series of steps between 408B and 408D may be repeated until a desired number of shape models remain in the grid of shape models.

After 408D, the method 400 may replace the image data with a higher-resolution version of the image data at 408E and attempt to fit the remaining shape models of the grid of shape models to the high-resolution image at 408F. Then, at 408G, the navigation computer 140 determines a cost function associated with each of the shape models. Like at 408C, the cost functions determined at 408G may be RMS residuals between the remaining shape models and the high-resolution image. After calculating the cost functions, the method 400 proceeds to discard at least one shape model of the remaining shape models based on the cost functions at 408H. The navigation computer 140 retains the shape models which most accurately represent the high-resolution image by retaining those with the lower cost functions.

Additionally or alternatively, after 408D, the method 400 may replace each of the remaining shape models with higher-resolution versions at 408E. In such an implementation, steps 408F through 408H may include attempting to fit the higher-resolution shape models to the image data at 408F, determining the cost functions associated with the higher-resolution shape models at 408G, and discarding the higher-resolution shape models that have the highest cost functions at 408H. The step 408E may even include replacing both of the image data and the remaining shape models with higher-resolution versions. In this case, steps 408F through 408H are carried out using the higher-resolution versions of the remaining shape models and the high-resolution image. Regardless of implementation, at 408I, the shape model with the lowest cost function is selected and the method 400 continues to 412.

Referring back to FIG. 8, the method 400 continues to step 412 after the multi-start algorithm has been carried out by the navigation computer 140 at 408. At 412, the navigation computer 140 attempts to determine a pose of each of the bones present in the image data. The bone poses may be determined based on the poses of the bones represented by the shape model selected at 408. It is also contemplated that the navigation computer 140 may use a neural network to detect the bones and bone poses, and/or other algorithms, such as random forests, support vector machines, and/or k-means. After determining the bone pose(s) at 412, the method 400 may continue to 416 to determine whether metal is present in the image data. The determination at 416 is similar to the determination at 317 of the previous method 300. If metal is detected at 416, the method 400 continues to the metal process at 418. The metal process carried out at 418 is like that shown in FIG. 4. More specifically, the metal process of the method 400 may include converting at least a portion of the image data into a binary image, along with fitting a binary shape model to the binary image(s) to create a metal segmentation. Alternatively, if metal is not detected, the method 400 proceeds to 420. Like for the previous method 300, the method 400 employs a first segmentation process at 420 as described below, including fitting the shape model selected by the multi-start algorithm to the image data, and this fitting process may not be reliable if metal is present in the image data.

At 420, the navigation computer 140 performs the first segmentation process on the image data. The first segmentation process includes generating a first segmentation of the bone(s) using the output of the multi-start algorithm, which includes the shape model selected by the algorithm. If the image data include more than one bone, such as a tibia and femur, the first segmentation process may include generating the first segmentation of the bones using two selected shape models, each of the two selected shape models corresponding to one of the bones. In this case, the multi-start algorithm may have been applied twice, first to select the shape model for the first bone, and second to select the shape model for the second bone. Alternatively, the selected shape model may include each of the bones included in the image data. At 424, the navigation computer 140 may perform a second segmentation process on the image data to refine the first segmentation. The second segmentation process includes generating a second segmentation of the vertebrae by providing two inputs to a neural network: the image data, and the first segmentation of the bone(s). Alternatively, the second segmentation process may refine the metal segmentation by generating the second segmentation of the bone(s) by providing the image data and the metal segmentation to the neural network. The neural network may be a convolutional neural network or any suitable alternative.

The remaining steps of the method 400, 428 through 440, are similar to the final series of steps of the previous method 300, 328 to 340. At 424, the navigation computer 140 fits the selected shape model(s) to the second segmentation, such as by morphing the selected shape model until it is substantially similar to the second segmentation. For example, if the method 400 is being carried out on a knee, the selected shape model may include a model tibia and model femur. This model tibia and model femur may be spatially morphed to be the same shape as the tibia and femur present in the image data. Subsequently, at 432, the navigation computer 140 applies landmarks (e.g. specific elements/surfaces of the bone(s), etc.) of the selected shape model to the second segmentation. The locations of the landmarks of the shape model are aligned with the locations of the landmarks in the second segmentation when the model is morphed to fit the second segmentation. At 432, like at 332, these landmark locations are applied to the second segmentation itself. For example, if the landmark is an element/surface of the knee described above, this knee landmark is applied to the second segmentation at substantially the same location as the corresponding element/surface of the knee. The selected shape model may include other landmarks which are also applied to the second segmentation. The method 400 then continues to 436.

At 436, the navigation computer 140 overlays a segmentation mask, the mask being based on the second segmentation, over the image data. This step 436 of the method 400 is similar to the step 336 of the previous method 300. As such, in some implementations, the navigation computer 140 may utilize an algorithm such as marching cubes or the like to convert the segmentation mask into a refined surface mesh. In such an implementation, the navigation computer 140 also projects/maps the shape model (and its corresponding information) onto the refined surface mesh to create a segmentation output. The segmentation output is thus based on the second segmentation and may further include the landmarks, planning information, and/or zones from the selected shape model. The segmentation mask may be a binary segmentation mask, a semantic segmentation mask, or a suitable alternative. The segmentation mask may be converted into a surface mesh that is aligned with the image data. In one implementation, the segmentation mask is converted into the surface mesh using a neural network and refined with a marching cubes algorithm. As a result, the user may view the surface mesh overlaid onto the image data with the GUI 150. Like the previous method 300, the segmentation mask generated during the method 400 may be based on both of the second segmentation and the metal segmentation. In this case, the navigation computer 140 still overlays the segmentation over the image data and the navigation computer 140 may still utilize an algorithm such as marching cubes or the like to convert the segmentation mask into the refined surface mesh. In such an implementation, the navigation computer 140 not only projects/maps the shape model (and its corresponding information) onto the refined surface mesh like in the above implementation, but also projects/maps the binary shape model (and its corresponding information) from the metal process onto the refined surface to create a segmentation output.

Finally, at 440, the method 400 may include an error handling process to determine whether any errors exist within the segmentation mask and/or whether any errors otherwise occurred during the segmentation method 400. The error handling process may be like the error handling process shown in FIG. 5. In some implementations, the error handling process of the method 400 does not include steps 340A or 340B, but otherwise includes step 340C.

The methods in accordance with the present teachings is for example a computer implemented method. For example, all the steps or merely some of the steps (i.e. less than the total number of steps) of the method in accordance with the present teachings can be executed by a computer (for example, at least one computer). A configuration of the computer implemented method is a use of the computer for performing a data processing method. Further, in the present teachings, the methods disclosed herein comprise executing, on at least one processor of at least one computer (for example at least one computer being part of the navigation system), the following exemplary steps which are executed by the at least one processor.

Several implementations have been discussed in the foregoing description. However, the implementations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

### Clauses for Additional Protection:

I. A method for performing segmentation on image data of a spine, the method comprising:
retrieving the image data of the spine, the image data including image data associated with a plurality of vertebrae;
detecting an estimated position of at least four pedicle regions for the image data associated with the plurality of vertebrae;
determining a pose for each of at least two of vertebra based on the detected estimated position of the at least four pedicle regions using on a multiple hypothesis approach;
performing a first segmentation process on the image data associated with the at least two vertebra with a shape model to generate a first segmentation of the at least two vertebra;
performing a second segmentation process on the image data associated with the at least two vertebra using a first neural network to generate a second segmentation of the at least two vertebra, the second segmentation process utilizing, as a first input, the image data associated with the at least two vertebra, and as a second input, the first segmentation of the at least two vertebra; and
overlaying a segmentation mask over the image data, the segmentation mask being based on the second segmentation.

II. A method for performing segmentation on image data of an anatomical region, the method comprising:
retrieving the image data of the anatomical region, the image data including image data associated with a plurality of anatomical objects;
detecting an estimated position of at least two regions of the plurality of anatomical objects for the image data;
determining a pose for each of at least two of anatomical objects based on the detected estimated position of the at least two regions using on a multiple hypothesis approach;
performing a first segmentation process on the image data associated with the at least two anatomical objects with a shape model to generate a first segmentation of the at least two anatomical objects;
performing a second segmentation process on the image data associated with the at least two anatomical objects using a first neural network to generate a second segmentation of the at least two anatomical objects, the second segmentation process utilizing, as a first input, the image data associated with the at least two anatomical objects, and as a second input, the first segmentation of the at least two anatomical objects; and
outputting an output being based on the second segmentation and the metal segmentation.

III. The method of clause II, further comprising
detecting an estimated position of at least four pedicle regions for the first image data associated with the plurality of vertebrae and the second image data associated with a least two of the plurality of vertebrae; and
determining a pose for each of at least two vertebrae based on the detected estimated position of the at least four pedicle regions using on a multiple hypothesis approach.

IV. The method of clause III, further comprising applying landmarks from the shape model to the output of the second segmentation process using the mapping.

V. The method of clause I, wherein the output is a segmentation mask.

VI. A method for performing segmentation on CT image data of a spine, the method comprising:
retrieving the CT image data of the spine, the CT image data including image data associated with a plurality of vertebrae;
performing a first segmentation process on the CT image data associated with at least two vertebrae of the plurality of vertebrae with a shape model to generate a first segmentation of the at least two vertebrae;
performing a second segmentation process on the CT image data associated with the at least two vertebrae using a first neural network to generate a second segmentation of the at least two vertebrae, the second segmentation process utilizing, as a first input, the CT image data associated with the at least two vertebrae, and as a second input, the first segmentation of the at least two vertebrae;
mapping the shape model to an output of the second segmentation process;
applying one or more points from the shape model to the output of the second segmentation process using the mapping; and
overlaying a segmentation mask over the CT image data, the segmentation mask being based on the second segmentation.

VII. The method of clause VI, further comprising:
detecting an estimated position of at least four pedicle regions for the CT image data associated with the plurality of vertebrae; and
determining a pose for each of at least two vertebrae based on the detected estimated position of the at least four pedicle regions using a multiple hypothesis approach.

VIII. The method of clause VI, wherein:
the shape model includes at least two model vertebrae, and
the step of performing the first segmentation process on the CT image data associated with the at least two vertebrae with the shape model to generate the first segmentation of the at least two vertebra includes associating each of the at least two vertebrae with a respective one of the at least two model vertebrae.

IX. The method of clause VIII, wherein the shape model further includes model pedicle positions associated with each of the at least two model vertebrae and each of the at least two vertebrae are associated with the respective one of the at least two model vertebrae based on a comparison of estimated positions of at least four pedicle regions and the model pedicle positions.

X. The method of clause IX, wherein the step of applying one or more points from the shape model to the output of the second segmentation process using the mapping; is further defined as applying one or more landmarks from the shape model to the output of the second segmentation process using the mapping, wherein the one or more landmarks are selected from the group consisting of a first pedicle, a second pedicle, a lamina, and a superior endplate.

XI. The method of clause VII, wherein the step of determining the pose of the at least two vertebra includes determining the pose of a first vertebra of the least two vertebrae relative to a reference coordinate space of the CT image data.

XII. The method of clause VII, wherein the step of determining a pose of the at least two vertebra includes determining a pose of a second vertebra of the at least two vertebrae relative to a position and orientation of the first vertebra of the at least two vertebrae.

XIII. The method of clause VII, wherein the multiple hypothesis approach is a Bayesian multiple hypothesis approach.

XIV. The method of clause XIII, wherein the step of determining the pose for at least two vertebrae based on the detected estimated position using the Bayesian multiple hypothesis approach includes generating a probability weighting graph.

XV. The method of clause VI, wherein the active appearance model is a binary active appearance model.

XVI. The method of clause XV, the method further comprising receiving user input with respect to an image region of the CT image data, the image region corresponding to one of the plurality of vertebrae.

XVII. The method of clause XVI, wherein the user input is indicative of a desired level label for one of the plurality of vertebrae.

XVIII. The method of clause XVII, further comprising selecting the shape model from a plurality of active appearance models based on the user input.

XIX. The method of clause XVIII, further comprising generating a plurality of additional labels, each one of the plurality of additional labels being associated with one of the plurality of vertebra of the CT image data.

XX. The method of clause VI, wherein the first neural network is a convolutional neural network.

XXI. The method of clause XX, wherein the second neural network is a convolutional neural network.

XXII. The method of clause VI, further comprising converting the segmentation mask to a surface mesh.

XXIII. The method of clause XXII, further comprising calculating anatomical information including a pose of at least one anatomical landmark based on the second segmentation.

XXIV. The method of clause XXIII, further comprising receiving user input with respect to the segmentation mask.

XXV. The method of clause XXIV, further comprising, in response to the user input with respect to the segmentation mask, indicating at least one of the segmentation is incorrect and a label associated with a vertebra is incorrect.

XXVI. A non-transitory computer readable storage medium having stored therein data representing instructions executable by a programmed processor for vertebra segmentation for three-dimensional computed tomography, the storage medium comprising instructions for:
retrieving CT image data of a spine, the CT image data including image data associated with a plurality of vertebrae;
performing a first segmentation process on the CT image data associated with at least two vertebrae of the plurality of vertebrae with a shape model to generate a first segmentation of the at least two vertebrae;
performing a second segmentation process on an image region of the CT image data associated with the at least two vertebrae using a first neural network to generate a second segmentation of the at least two vertebrae, the second segmentation process utilizing, as a first input, the CT image data associated with the at least two vertebrae, and as a second input, the first segmentation of the at least two vertebrae; and
overlaying a segmentation mask over the CT image data, the segmentation mask being based on the second segmentation.

XXVII. The non-transitory computer readable storage medium of clause XXVI, the medium further comprising instructions for:
detecting an estimated position of at least four pedicle regions for the CT image data associated with the plurality of vertebrae; and
determining a pose of each of the at least two of vertebra based on the detected estimated position of the at least four pedicle regions using a multiple hypothesis approach.

XXVIII. A method for performing segmentation on CT image data of a spine, the method comprising:
retrieving the CT image data of the spine, the CT image data including a first image data associated with a plurality of vertebrae and a second image data associated with at least two of the plurality of vertebrae;
determining that metal is present in the second image data and employing a metal process in response to determining that metal is present in the second image data, the metal process including:
converting at least a portion of the second image data to at least one binary image,
generating a metal segmentation of the at least two of the plurality of vertebrae using a neural network, and
fitting a binary shape model to the at least one binary image; and
optionally, overlaying a first segmentation mask over the CT image data, the first segmentation mask being based on the metal segmentation.

XXIX. The method of clause XXVIII, further comprising:
applying landmarks from the binary shape model to the first segmentation mask using the fitting of the binary of the shape model.

XXX. The method of clause XXVIII, further comprising:
performing a first segmentation process on the first image data with a second shape model to generate a first segmentation of the at least two vertebrae;
performing a second segmentation process on the first image data using a first neural network to generate a second segmentation of the at least two vertebrae, the second segmentation process utilizing, as a first input, the first image data, and as a second input, the first segmentation of the at least two vertebrae;
mapping the second shape model to the output of the second segmentation process;
applying landmarks from the second shape model to the output of the second segmentation process using the mapping; and
overlaying a second segmentation mask over at least a portion of the CT image data, the second segmentation mask being based on the second segmentation.

XXXI. The method of clause XXVIII, further comprising:
detecting an estimated position of at least four pedicle regions for the first image data associated with the plurality of vertebrae and the second image data associated with a least two of the plurality of vertebrae;
determining a pose for each of at least two vertebrae based on the detected estimated position of the at least four pedicle regions using a multiple hypothesis approach;
performing a first segmentation process on the first image data with a shape model to generate a first segmentation of the at least two vertebrae;
performing a second segmentation process on the first image data using a first neural network to generate a second segmentation of the at least two vertebrae, the second segmentation process utilizing, as a first input, the first image data, and as a second input, the first segmentation of the at least two vertebrae;
mapping the shape model to the output of the second segmentation process;
applying landmarks from the shape model to the output of the second segmentation process using the mapping; and
overlaying a second segmentation mask over at least a portion of the CT image data, the second segmentation mask being based on the second segmentation.

XXXII. A method for performing segmentation on CT image data, the method comprising:
retrieving the CT image data, the CT image data including a first image data associated with an anatomical element of a patient and a second image data associated with at least a portion of the anatomical element;
determining that metal is present in the second image data and employing a metal process in response to determining that metal is present in the second image data, the metal process including:
converting at least a portion of the second image data from a CT image format to at least one binary image, and
generating a metal segmentation of the second image data using a neural network; and
fitting a binary shape model to the at least one binary image;
overlaying a first segmentation mask over the CT image data, the first segmentation mask being based on the metal segmentation.

XXXIII. The method of clause XXXII, wherein the step of overlaying the first segmentation mask over the CT image data includes overlaying the first segmentation mask over the second image data.

What is claimed is:

1. A method for performing segmentation on image data including a first bone, the method comprising:
retrieving the image data of the first bone;
performing a first segmentation process on the image data associated with the first bone with a first shape model to generate a first segmentation of the first bone;
performing a second segmentation process on an image region of the image data associated with the first bone using a first neural network to generate a second segmentation of the first bone, the second segmentation process utilizing, as a first input, the image data associated with the first bone, and as a second input, the first segmentation of the first bone;
mapping the output of the first shape model to an output of the second segmentation process; and
determining the anatomical landmarks in the output of the second segmentation based on the mapping.

2. The method of claim 1, further comprising:
detecting an estimated position of at least one region or feature of the first bone in the image data; and
determining a pose for the first bone based on the detected estimated position of the at least one region or feature.

3. The method of claim 1, further comprising applying anatomical landmarks from the first shape model to the output of the second segmentation process using the mapping.

4. The method of claim 1, wherein the first bone is a femur, a tibia, a pelvis, or a vertebra.

5. The method of claim 1, further comprising:
retrieving image data of a second bone;
performing a third segmentation process on the image data associated with the second bone with a second shape model to generate a first segmentation of the second bone;
performing a fourth segmentation process on the image region of the image data associated with the second bone using a second neural network to generate a second segmentation of the second bone, the fourth segmentation process utilizing, as a first input, the image data associated with the second bone, and as a second input, the first segmentation of the second bone;
mapping the second shape model to an output of the fourth segmentation process for the second bone; and
displaying the output of the fourth segmentation process.

6. The method of claim 5, wherein the first bone is a femur, and the second bone is a tibia.

7. The method of claim 5, wherein the first bone is a first vertebra, and the second bone is a second vertebra different from the first vertebra.

8. The method of claim 5, wherein the first shape model is different from the second shape model, and the first neural network is different from the second neural network.

9. The method of claim 5, further comprising generating a plurality of labels, each one of the plurality of labels being associated with one of the first bone and the second bone.

10. The method of claim 9, wherein the step of generating the plurality of labels is based on the first shape model and the second shape model.

11. The method of claim 1, wherein the first shape model is further defined as a grid of a plurality of active appearance model instances and the method further comprises:
running each active appearance model instance against the image data;
culling at least one instance from the plurality of run active appearance model instances based on a cost associated with each active appearance model instance; and
performing the first segmentation process using the at least one of the active appearance model instances that remain after the step of culling at least one instance.

12. A non-transitory computer readable storage medium having stored therein data representing instructions executable by a programmed processor for bone segmentation for three-dimensional computed tomography, the storage medium comprising instructions for:
retrieving image data of a first bone;
performing a first segmentation process on the image data associated with the first bone with a first shape model to generate a first segmentation of the first bone;
performing a second segmentation process on an image region of the image data associated with the first bone using a first neural network to generate a second segmentation of the first bone, the second segmentation process utilizing, as a first input, the image data associated with the first bone, and as a second input, the first segmentation of the first bone;
mapping the first shape model to an output of the second segmentation process; and
displaying the output of the second segmentation process.

13. The non-transitory computer readable storage medium of claim 12, the medium further comprising instructions for:
detecting an estimated position of at least one region or feature of the first bone in the image data; and
determining a pose for each of the first bone based on the detected estimated position of the at least one region or feature.

14. The non-transitory computer readable storage medium of claim 12, the medium further comprising instructions for applying landmarks from the first shape model to the output of the second segmentation process using the mapping.

15. The non-transitory computer readable storage medium of claim 12, the medium further comprising instructions for:
detecting an estimated position of at least four pedicle regions of the image data associated with a plurality of vertebrae; and
determining a pose for each of at least two vertebrae based on the detected estimated position of the at least four pedicle regions using a multiple hypothesis approach.

16. The non-transitory computer readable storage medium of claim 12, wherein the first bone is a femur, a tibia, a pelvis, or a vertebra.

17. The non-transitory computer readable storage medium of claim 12, the medium further comprising instructions for:
retrieving image data of a second bone;
performing a third segmentation process on the image data associated with the second bone with a second shape model to generate a first segmentation of the second bone;
performing a fourth segmentation process on the image region of the image data associated with the second bone using a second neural network to generate a second segmentation of the second bone, the fourth segmentation process utilizing, as a first input, the image data associated with the second bone, and as a second input, the first segmentation of the second bone;
mapping the second shape model to an output of the fourth segmentation process for the second bone; and
displaying the output of the fourth segmentation process.

18. The non-transitory computer readable storage medium of claim 17, wherein the first bone is a femur, and the second bone is a tibia.

19. The non-transitory computer readable storage medium of claim 17, wherein the first bone is a first vertebra, and the second bone is a second vertebra different from the first vertebra.

20. The non-transitory computer readable storage medium of claim 17, wherein the first shape model is different from the second shape model, and the first neural network is different from the second neural network.

21. The non-transitory computer readable storage medium of claim 12, wherein the first shape model is further defined as a grid of a plurality of active appearance model instances and the medium further includes instructions for:
running each active appearance model instance against the image data;
culling at least one instance from the plurality of run active appearance model instances based on a cost associated with each active appearance model instance; and
performing the first segmentation process using the at least one of the active appearance model instances that remain after the step of culling at least one instance.
